(19) United States
(12) Patent Application Publication
(10) Pub. No.: US 2018/0355432 A1
(43) Pub. Date: Dec. 13, 2018

(54) IDENTIFICATION OF EPILEPSY PATIENTS AT INCREASED RISK FROM SUDDEN UNEXPECTED DEATH IN EPILEPSY

(71) Applicant: New York University, New York, NY (US)

(72) Inventors: Matija SNUDERL, New York, NY (US); Kasthuri KANNAN, Fairlawn, NJ (US); Daniel FRIEDMAN, Montclair, NJ (US); Orrin DEVINSKY, New York, NY (US)

(21) Appl. No.: 15/781,276

(22) PCT Filed: Dec. 5, 2016
(86) PCT No.: PCT/US2016/064970

§ 371 Date: Jun. 4, 2018

Related U.S. Application Data
(60) Provisional application No. 62/263,078, filed on Dec. 4, 2015.

(51) Int. Cl. C12Q 1/6883; A61B 5/00

(52) U.S. Cl. CPC C12Q 1/6883; A61B 5/4094; G16H 50/20; C12Q 2600/156; C12Q 2600/158

(57) ABSTRACT

Provided is a method for predicting an individual to be at risk of developing sudden unexpected death in epilepsy (SUDEP) comprising determining the presence or absence of mutations in the genes ITPR1, GABRR2, JUP, SSTR5, F2, KCNMB1, CNTNAP2, GRM8, GNAI2, TUBA3D, GRIK1, GRIK5 and DPP6, or determining if the expression of certain cardiac arrhythmia genes or gamma-aminobutyric acid/glutamate metabolism genes are increased or decreased.

### CROSS-REFERENCE TO RELATED APPLICATIONS

This application claims priority to U.S. Provisional application No. 62/263,078, filed on Dec. 4, 2015, the disclosure of which is incorporated herein by reference.

### BACKGROUND OF THE DISCLOSURE

Sudden Unexpected Death in Epilepsy (SUDEP) is said to occur when a person with epilepsy dies unexpectedly and was previously in a usual state of health. The death is not known to be related to an accident or seizure emergency such as status epilepticus. When an autopsy is done, no structural or toxicological cause of death can be found. Each year, more than 1 out of 1,000 people with epilepsy die from SUDEP. However, it occurs more frequently in people with epilepsy whose seizures are poorly controlled. One out of 150 people with poorly controlled epilepsy may die from SUDEP each year. SUDEP takes more lives annually in the United States than sudden infant death syndrome (SIDS). Most importantly, SUDEP is the leading cause of death in young people with certain types of uncontrolled epilepsy. The causes of SUDEP are not known. SUDEP occurs most often at night or during sleep and the death is not witnessed, leaving many questions unanswered. Currently no laboratory tests that could help identify patients at risk of SUDEP.

### SUMMARY OF THE DISCLOSURE

This disclosure is based on identification that patients who died of SUDEP had unique genetic signature compared to epilepsy patients who did not die of SUDEP. Specific mutations involved GABA/Glutamate receptor signaling pathway and cardiac arrhythmia genes were identified. Further, expression of several genes was found to be enhanced or reduced in the brains of patients who died of SUDEP as compared to epilepsy patients who did not die of SUDEP or normal individuals. Based on these observations, the present disclosure provides methods for predicting likelihood of epilepsy patients progressing to SUDEP. The method comprises identifying the presence of one or more specific mutations described herein, or determining if the expression of one or more genes disclosed herein is increased or decreased as compared to controls. An increase in the expression of certain genes, or the decrease in the expression of certain genes is predictive of a likelihood that the individual will progress to SUDEP. Based on such identification, the individual can be monitored and treated.

### BRIEF DESCRIPTION OF THE DRAWINGS

FIG. 1 is a representation of the exome bioinformatics analysis.

FIG. 2 is an overview of the exome bioinformatics analysis.

FIG. 3 is a representation of mutations identified in SUDEP patients.

FIG. 4 is a representation of mutations shared between SUDEP and Control epilepsy cohort. The genes on which the mutations are present are indicated.

FIG. 5 is a representation of targeted RNA seq analysis of mutated genes. X=mutation identified in that gene

FIG. 6 shows a comparison of SUDEP patients who had mutations in genes of glutamate/GABA signaling (S GL/GA) vs all control patients (C), patients who suffered from epilepsy but did not die of SUDEP.

FIG. 7 shows comparison of SUDEP patients who had mutations in cardiac (S CARDIO) vs all control patients (C), patients who suffered from epilepsy but did not die of SUDEP.

### DESCRIPTION OF THE DISCLOSURE

This disclosure provides identification of a unique genetic pattern in patients who died of SUDEP. Mutations involved GABA/Glutamate receptor signaling pathway or cardiac arrhythmia genes. These mutations were not present in age/sex matched controls of patients with epilepsy who are alive. Nor were they present in other public genomic databases such as 1) dbSNP, 2) 1000 genomes, 3) ESP6500 exome database or epilepsy SPECIFIC CarpeDB database. Based on the data provided herein, it is considered that these mutations are strongly associated with the SUDEP phenotype. Such mutations are termed herein as SUDEP specific mutations. The mutation spectrum provided in this disclosure is relatively specific for the SUDEP population and therefore, provides relevant biomarkers.

In one aspect, this disclosure provides an in vitro method for identifying, or aiding in identifying, predicting, or aiding in predicting, a human individual as being at risk of developing SUDEP comprising detecting in a test sample derived from the individual one or more SUDEP specific mutations in one or more marker genes. For example, the method comprises identifying, or aiding in identifying, predicting, or aiding in predicting an individual (such as an individual who is suffering from, or has been diagnosed with epilepsy) as being at risk of developing SUDEP comprising detecting in a test sample obtained from the individual one or more SUDEP specific mutations in one or more marker genes selected from the group consisting of: ITPR1, GABRR2, JUP, SSTR5, F2, KCNMB1, CNTNAP2, GRM8, GNAI2, TUBA3D, GRIK1, GRIK5 and DPP6. For example, mutations may be detected in the group of genes involved in GABA/Glutamate receptor signaling pathway and/or cardiac arrhythmia genes. For example, mutations may be present in one or more of ITPR1, GABRR2, SSTR5, CNTNAP2, GRM8, GNAI2, GRIK1, and GRIK5 (GABA/Glutamate receptor signaling pathway genes), and/or they may be present in JUP, F2, KCNMB1, TUBA3D, and DPP6 (cardiac arrhythmia genes). Mutations may be present in two or more of the GABA/Glutamate receptor signaling pathway genes, or two or more of the cardiac arrhythmia genes. In certain SUDEP patients, mutations were observed in SSTR5 and GRIK1, GRM8 and GNAI2, and TUBA3D, F2 and JUP. Some or all of the mutations shown in Tables 1 and 2 can be detected. For example, 1, 2, 3, 4, 5, 6, 7, 8, 9, 10, 11, 12 or 13 gene mutations shown in Tables 1 and 2 can be detected. The GABA/Glutamate receptor signaling pathway gene mutations can be tested separately from the gene mutations in the cardiac arrhythmia genes or they can all be tested together.

The test sample for testing can be obtained from an individual. Typically, the individual will have been diagnosed with epilepsy. The test samples may include body tissues (e.g., biopsies or resections) and fluids, such as blood, sputum, cerebrospinal fluid, and urine. The test samples may contain a single cell, a cell population (i.e. two or more cells) or a cell extract derived from a body tissue. The test samples are generally collected in a clinically acceptable manner, in a way that nucleic acids and/or proteins are preserved so that they can be detected. The test samples may be used in unpurified form or subjected to enrichment or purification step(s) prior to use, for example in order to isolate the DNA, RNA or the protein fraction in a given sample. Such techniques are known to those skilled in the art. (See, e.g., Sambrook, J., and Russel, D. W. (2001), Molecular cloning: A laboratory manual (3rd Ed.) Cold Spring Harbor, N.Y., Cold Spring Harbor Laboratory Press; Ausubel, F. M. et al. (2001) Current Protocols in Molecular Biology, Wiley & Sons, Hoboken, N.J., USA).

Suitable techniques for determining the presence or absence of the mutations include but are not limited to sequencing methodologies, hybridization of probes or primers directed to genomic DNA or cDNA, and/or by using various chip technologies, polynucleotide or oligonucleotide arrays, and combinations thereof. Thus, probes directed to polynucleotides comprising the mutations can be arranged and/or fixed on a solid support. For amplification or sequencing reactions, primers can be designed which hybridize to a segment of a polynucleotide comprising or proximal to the mutations and used to obtain nucleic acid amplification products (i.e., amplicons). Those skilled in the art will recognize how to design suitable primers and perform amplification and/or hybridization and/or sequencing reactions in order to carry out various embodiments of the method. The primers/probes can comprise modifications, such as being conjugated to one or more detectable labels.

In one embodiment, the method comprises determining in a test sample obtained from an individual, such as an individual who has been diagnosed with epilepsy, one or more of the mutations in Table 1.

TABLE 1

| Gene | Accession no. and mutation |
|---|---|
| ITPR1 | NM_002222:exon40:c.G5278A:p.A1760T, |
| | NM_001099952:exon41:c.G5323A:p.A1775T |
| | NM_001168272:exon43:c.G5422A:p.A1808T |
| GABRR2 | NM_002043:exon4:c.G352T:p.A118S |
| JUP | NM_002230:exon4:c.A493G:p.I165V |
| | NM_021991:exon4:c.A493G:p.I165V |
| SSTR5 | NM_001053:exon1:c.G994T:p.A332S |
| | NM_001172560:exon2:c.G994T:p.A332S |
| F2 | NM_000506:exon11:c.C1435T:p.H479Y |
| KCNMB1 | NM_004137:exon4:c.T530C:p.M177T |
| CNTNAP2 | NM_014141:exon13:c.G2047A:p.E683K |
| GRM8 | NM_001127323:exon2:c.A247G:p.I83V |
| | NM_000845:exon1:c.A247G:p.I83V |
| GNAI2 | NM_001282620:exon1:c.C65T:p.S22F |
| TUBA3D | NM_080386:exon4:c.A554G:p.Y185C |
| GRIK1 | NM_000830:exon7:c.A988G:p.M330V |
| | NM_175611:exon7:c.A988G:p.M330V |
| GRIK5 | NM_002088:exon17:c.T2273A:p.F758Y |
| DPP6 | NM_130797:exon1:c.C160G:p.R54G |

The different GenBank accession numbers for a gene refer to splice variants. The single nucleotide polymporphisms (SNP) for a gene has the same location on the chromosome, but may manifest itself as different location on the mRNA due to splice variants as indicated by reference to the cDNAs in Table 1.

After variants chromosome and coordinates are obtained, the annotation for the gene, cDNA and amino acid change were obtained by running the coordinate file through the program called ANNOVAR. ANNOVAR is an efficient software tool to utilize up-to-date information to functionally annotate genetic variants detected from diverse genomes. Given a list of variants with chromosome, start position, end position, reference nucleotide and observed nucleotide, ANNOVAR can perform gene based annotation that can identify whether a variant cause protein coding changes in the genome through the amino acids that are affected.

A 200 nucleotide sequence containing each of the mutations is provided in the following SEQ IDs shown in Table 2 for each SUDEP gene mutation.

TABLE 2

| Gene/Location | SEQ ID |
|---|---|
| ITPR1::chr3:4776861-4777061 | SEQ ID NO: 1 |
| GABRR2::chr6:89978790-89978990 | SEQ ID NO: 2 |
| JUP::chr17:39925335-39925535 | SEQ ID NO: 3 |
| SSTR5::chr16:1129762-1129962 | SEQ ID NO: 4 |
| F2::chr11:46750250-46750450 | SEQ ID NO: 5 |
| KCNMB1::chr5:169805654-169805854 | SEQ ID NO: 6 |
| CNTNAP2::chr7:147336247-147336447 | SEQ ID NO: 7 |
| GRM8::chr7:126882912-126883112 | SEQ ID NO: 8 |
| GNAI2::chr3:50264520-50264720 | SEQ ID NO: 9 |
| TUBA3D::chr2:132237720-132237920 | SEQ ID NO: 10 |
| GRIK1::chr21:31015156-31015356 | SEQ ID NO: 11 |
| GRIK5::chr19:42507726-42507926 | SEQ ID NO: 12 |
| DPP6::chr7:153749965-153750165 | SEQ ID NO: 13 |

The mutation in each sequence ID in the following cDNA sequences is shown as bolded and underlined.

(SEQ ID NO: 1)
TCCAGCTCTATGAGCAGGGGTGAGATGAGTCTGGCCGAGGTTCAGT
GTCACCTTGACAAGGAGGGGGCTTCCAATCTAGTTATCGACCTCAT
CATGAACGCATCCAGTGACCGAGTGTTCCATGAAAGCATTCTCCTG
GCCATTGCCCTTCTGGAAGGAGGCAACACCACCATCCAGGTAGGAA
GGCAGCTTGGCTACTG
(SEQ ID NO: 2)
TGTCATGAGTGAACGATCTTTTGGAGTGAACAAAGAAGACATCAGG
GACCCAGATCTTCTTCACCAGCCGGCCATCGAAGGTCATGCTCTTG
TTGCTGGCGCTGGAGAAAGCTAGCCTCTCATCCTTCCAGTAATGCC
GCAGGTACAGGGTCATAGTGAAGTCCTGTGGGAGCCGGGGTGAGAC
CAGACAAAAATGGCTT
(SEQ ID NO: 3)
CGCTGGTATTCTGCATGGTACGCACGACAGCGGCCACCAGCTGGGG
CGAGCCCATCAGGGCCCGCCGCGACGCCTCCTTCTTCGACAGCTGG
TTCACAATCATGGCCGCCTTGGTCACCACCACCTGGAGGGCAAAGG
CAGGGGCGGGGACGTGAGCACTAAGGAGAGGCCGGGATACCCTTCC
ACAGAGCTGAGGAGGG
(SEQ ID NO: 4)
GCCAACCCCGTCCTCTACGGCTTCCTCTCTGACAACTTCCGCCAGA
GCTTCCAGAAGGTTCTGTGCCTCCGCAAGGGCTCTGGTGCCAAGGA
CGCTGACGCCACGGAGCCGCGTCCAGACAGGATCCGGCAGCAGCAG
GAGGCCACGCCACCCGCGCACCGCGCCGCAGCCAACGGGCTTATGC
AGACCAGCAAGCTGTG
(SEQ ID NO: 5)
AAGATCTACATCCACCCCAGGTACAACTGGCGGGAGAACCTGGACC
GGGACATTGCCCTGATGAAGCTGAAGAAGCCTGTTGCCTTCAGTGA
CTACATTCACCCTGTGTGTCTGCCCGACAGGGAGACGGCAGCCAGG
TGGGCCACCAGATGCTTGTTAGCTGAGGGGCAGAAGCCAAGTTCTG
GGCCTGGCTCTGATAC
(SEQ ID NO: 6)
GGCCCAGCCAGTCCCCTGTGCCCTGACAAGTGGTATGGCATGGATG
GATGGCTCTACTTCTGGGCCGCCAGGATGGACAGGTACTGGTTGCT
CTTCACCATGGCGATAATGAGGAGGCCACCGGTCAGCAGGAAGGTG
GGCCAGAAGAGGGAGAAGAGGAGGGCCTGGGGCCCGTAGAGGCGCT
GGAATAGGACGCTGGT
(SEQ ID NO: 7)
GTGGTCGGCTACAACCCAGAAAAATACTCAGTGACACAGCTCGTTT
ACAGCGCCTCCATGGACCAGATAAGTGCCATCACTGACAGTGCCGA
GTACTGCGAGCAGTATGTCTCCTATTTCTGCAAGATGTCAAGATTG
TTGAACACCCCAGGTAGGCTGAGAATGGAATGTTACTTTTAATCAC
TATCTCAGCTGGTGCT
(SEQ ID NO: 8)
ACTGCTCCAAAGCATAGGTGTCCCTAGAGCACGTGTCGAGGATGCG
GACACCCAGAGTGATGTTGGAAAGGAGATCAGGGTCCTTGTTAATC
TGGTCAATTGCATAAAGCATGGCCTCCAGTCTGTGAATCCCCTTTT
CCTTCTTCAGCTCCCCACAAGGCACCCCTCTCTCTCCCTTTGCGTG
GACAGGGAAGAGACCC
(SEQ ID NO: 9)
TGTGAAGTGGAAGCGCGAGAAGGAGGGAGCGTCTCATGACGGAGGG
TGTGAAGACGCTAGGCTGGACGAAGCAGAAAGGCGGGTGTCACTGG
GGACGTTCTGAGGGTAAGCCGATGGCGGCTATCGCGGAGGAGACCC
TGGCGAGGTGGGGCCCCGCGCGGGGCAAGGGGGATGGGGTGCCACA
GAGGGCTAGTTGCAAG
(SEQ ID NO: 10)
TGCTCATGGAGCGGCTCTCAGTGGATTACGGCAAGAAGTCCAAGCT
AGAGTTTGCCATTTACCCAGCCCCCCAGGTCTCCACAGCCGTGGTG
GAGCCCTACAACTCCATCCTGACCACCCACACGACCCTGGAACATT
CTGACTGTGCCTTCATGGTCGACAATGAAGCCATCTATGACATATG
TCGGCGCAACCTGGAC
(SEQ ID NO: 11)
GGTTCATAAATCTGGGTCCGAGGCGCCATGGCTTATGTCTATGGCA
CTGCAGGGAGCTGACGGTCAGCTGGGATGCCCGGTGCGAGGCAATG
GCCACCATGTACACAGCATCGTACATCAGAGCCGCTTCAGTCTGTG
GAGGAAAACACACACCGCATCTTAAATTCCACTTTTGCTTACCTTC
CTTTACTTGCATAATC
(SEQ ID NO: 12)
GGAAGGCCGTGGTGAGGGCACAGTTTGGGGTTTGGGGCGGTCAGGG
CTGCAGGGCCCGATGGCTGGTCCAGCCCCTCGTGTGCCTGCCCAGG
CTCCCCGTTCCGGGATGAGATCACACTGGCCATCCTGCAGCTTCAG
GAGAACAACCGGCTGGAGATCCTGAAGCGCAAGTGGTGGGAGGGGG
GCCGGTGCCCCAAGGA
(SEQ ID NO: 13)
CCCCCGGAGGCGAGTCACCTCCTGGGCGGCCAGGGGCCCGAGGAGG
ACGGCGGCGCAGGAGCCAAGCCCCTCGGCCCGCGGGCGCAGGCGGC
GGCGCCCCGGGAGCGCGGCGGCGGCGGCGGCGGCGCGGGTGGCCGG
CCCCGGTTCCAGTACCAGGCGCGGAGCGATGGTGACGAGGAGGACG
TAAGAGCTTCTCGGGG

The specific mutations can be: for ITPR1 gene, a change of G to A at position corresponding to position no. 100 of SEQ ID NO: 1; for GABRR2 gene, a change of G to T at position corresponding to position no. 100 of SEQ ID NO: 2; for JUP, a change of A to G at position corresponding to position no. 100 of SEQ ID NO: 3; for SSTR5, a change of G to T at position corresponding to position no. 100 of SEQ ID NO: 4; for F2, a change of C to T at position corresponding to position no. 100 of SEQ ID NO: 5; for KCNMB1, a change of T to C at position corresponding to position no. 100 of SEQ ID NO: 6; for CNTNAP2, a change of G to A at position corresponding to position no. 100 of SEQ ID NO: 7; for GRM8, a change of A to G at position corresponding to position no. 100 of SEQ ID NO: 8; for GNA12, a change of C to T at position corresponding to position no. 100 of SEQ ID NO: 9; for TUBA3D, a change of A to G at position no. 100 of SEQ ID NO: 10; for GRIK1, a change of A to G at position corresponding to position no. 100 of SEQ ID NO: 11; for GRIK5, a change of T to A at position corresponding to position no. 100 of SEQ ID NO: 12; and/or for DPP6, a change of C to G at position corresponding to position no. 100 of SEQ ID NO: 13.

The mutation also includes a mutation in the complementary nucleotide in the opposite strand. Based on the mutations and the chromosomal locations, one skilled in the art can design appropriate primers for identifying their presence. The sequences are provided here for convenience, however, sequence information can be obtained by one skilled in the art from the chromosomal locations and other information provided herein.

In one aspect, this disclosure provides a method for identifying SUDEP specific mutations. A SUDEP specific mutation is defined as a mutation (such as an SNP) which is identified as present in chromosomal DNA of individuals who died from SUDEP, but is absent in the chromosomal DNA of age/sex matched individuals who have epilepsy, but who, without intervention, did not die from SUDEP. To identify SUDEP specific mutations, test samples may be obtained from individuals who died from SUDEP and compared to their matched controls to identify SUDEP specific mutations as further described in the example below.

For example, while certain SUDEP specific mutations are described in Table 1 and in SEQ IDs 1-13 above, other SUDEP mutations in these genes or in other genes may be identified.

In one aspect, this disclosure provides a method for predicting an individual to be at risk of developing SUDEP comprising contacting a DNA or RNA from a test sample from the individual with a gene chip, wherein the gene chip comprises one or more probes that can detect one or more mutations in the genes specified in Table 1. For example, the one or more probes may detect one or more SNPs listed in Table 1, or as shown in SEQ IDs 1-13. The DNA may be cDNA or may be RNA, or amplified from chromosomal DNA, or whole genome sequencing, or transcriptome sequencing.

In one aspect, this disclosure provides a panel of probes, said panel comprising probes which can detect one or more mutations provided in Table 1. The panel may be in the form of a chip. Further, DNA microarrays can be used comprising polynucleotide probes, wherein the probes are designed to discriminate mutations, such as SNPs that are associated with SUDEP as described herein. For any single or any combination of the markers set forth in this disclosure, a DNA array or any chip or bead format for testing a plurality of polynucleotides can be provided. Various reagents, devices and procedures which comprise polynucleotide arrays and are used for analyzing nucleic acid samples are known in the art, are commercially available and can be adapted for use with the present disclosure. For instance, devices and services sold under the trade names ILLUMINA and AFFYMETRIX can be adapted to test biological samples obtained or derived from individuals for any one or any combination of the markers discussed herein, given the benefit of this disclosure. The disclosure includes determining heterozygous and homozygous mutations.

Any of the DNA sequences presented herein and any combination of them can be used in a DNA array on a chip. “DNA array” and “chip” are not intended to be limited to any particular configuration, and include all devices, articles of manufacture and processes that are used for concurrent testing of a plurality of distinct nucleic acids to determine multiple distinct SNPs present in the distinct polynucleotides.

Genomics analysis can be carried out such as chromosomal analysis whole genome sequencing, partial genome sequencing, transcriptome analysis, copy number variation analysis, and single nucleotide polymorphism (SNP) analyses. Genomics analysis may be carried out with an assay such as, for example, fluorescent in situ hybridization (FISH), comparative genome hybridization (CGH), polymerase chain reaction (PCR), semi-quantitative real-time PCR, multiplex PCR, oligonucleotide or nucleotide arrays, antibody arrays, and chromatin immunoprecipitation. Genomic analyses and assays may be applied to both genomic DNA and genomic RNA.

In one embodiment, instead of detecting mutated polynucleotides, the method comprises detecting mutated proteins encoded by any of the genes described in Tables 1 and 2. The detection can be carried out by using any suitable technique or reagent, and will generally entail separating the protein from a biological sample and reacting the separated protein with at least one specific binding partner. Such binding partners can include but are not necessarily limited to antibodies, whether polyclonal or monoclonal, and antibody fragments that can specifically bind to the protein, such as Fab fragments, Fab′ fragments, F(ab′)2 fragments, Fd fragments, Fv fragments, and scFv fragments. Other specific binding partners can include aptamers, diabodies, or any other reagent that can specifically recognize the mutant protein. Detecting a complex of a specific binding partner and mutant protein can be performed using any suitable technique, including Western blotting, and other immunodetection methods, such as enzyme linked immunosorbant assay (ELISA), a lateral flow test, etc.

In one embodiment, any mutations in DNA that result in the amino acid changes disclosed here may be identified and such mutations can be used as being predictive of the risk of developing SUDEP.

Once individuals are identified as being at risk of developing SUDEP, they can be provided a focused approach to prevent SUDEP. Since some of genes identified in the present disclosure are associated with cardiac arrhythmia, individuals that are identified as containing one or more mutations in genes associated with cardiac arrhythmia, these patients can be followed up with cardiac evaluation to rule out cardiac arrhythmia and/or treat it if present. Furthermore, these patients could take additional precautions such as making sure that their seizures are under control (uncontrolled seizures are one of the risk factors of SUDEP), take medications regularly, visit healthcare team regularly especially if seizures are not controlled well, strongly avoid potential seizure triggers such as alcohol, recreational drugs. Patients would take extra effort to make sure family and coworkers know what to do for seizure first-aid, take extra precautions around water, including swimming and bathing.

Once specific mutations are identified in individuals, therapeutic approach could be tailored to fit best the patient's mutation status to prevent death from SUDEP. For example, a patient with a mutation in the GABA/Glutamate pathway may benefit from a GABA targeting therapy including: 1) GABA Receptor Agonists such as benzodiazepines, barbiturates, and other substances such as picrotoxins, bicuculline, and neurosteroids; 2) GABA reuptake inhibitors such as Nipecotic acid and tiagabine; 3) GABA Transaminase inhibitiors such as Vigabatrin, or Glutamate targeting therapy such as Glutamate blockers including felbamate, Topiramate, Perampanel. In patients with mutation in the KCNMB1 gene, which is a potassium calcium-activated channel subfamily M regulatory beta subunit 1 gene, an anti-epilepsy medication targeting potassium channels such as Ezogabine (Potiga), known as retigabine, may be administered for seizure control.

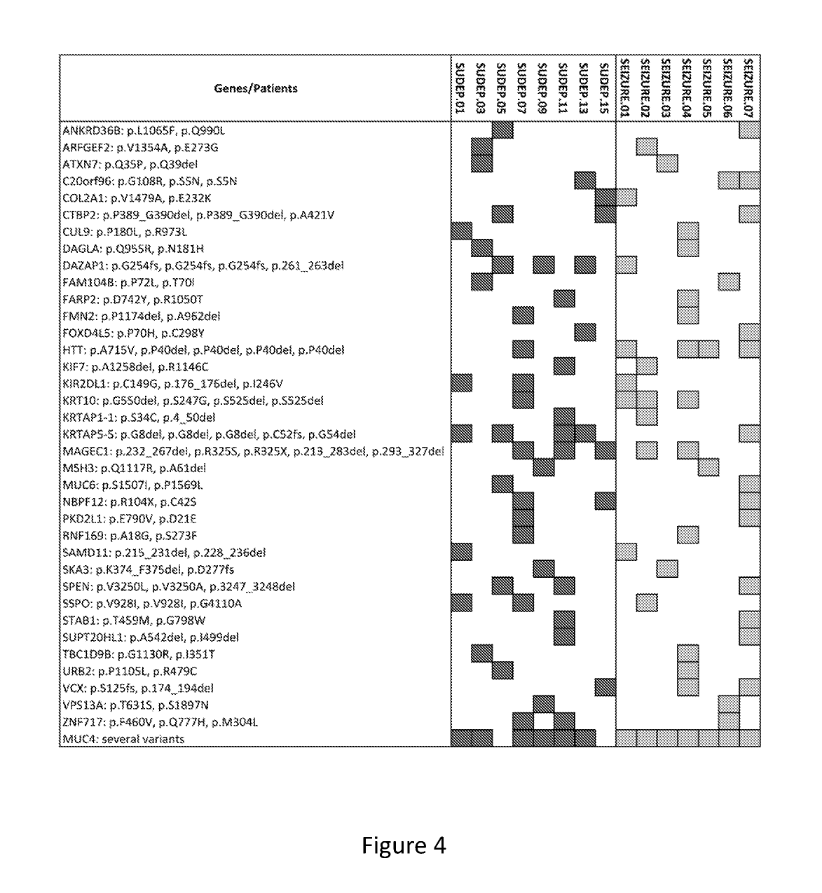

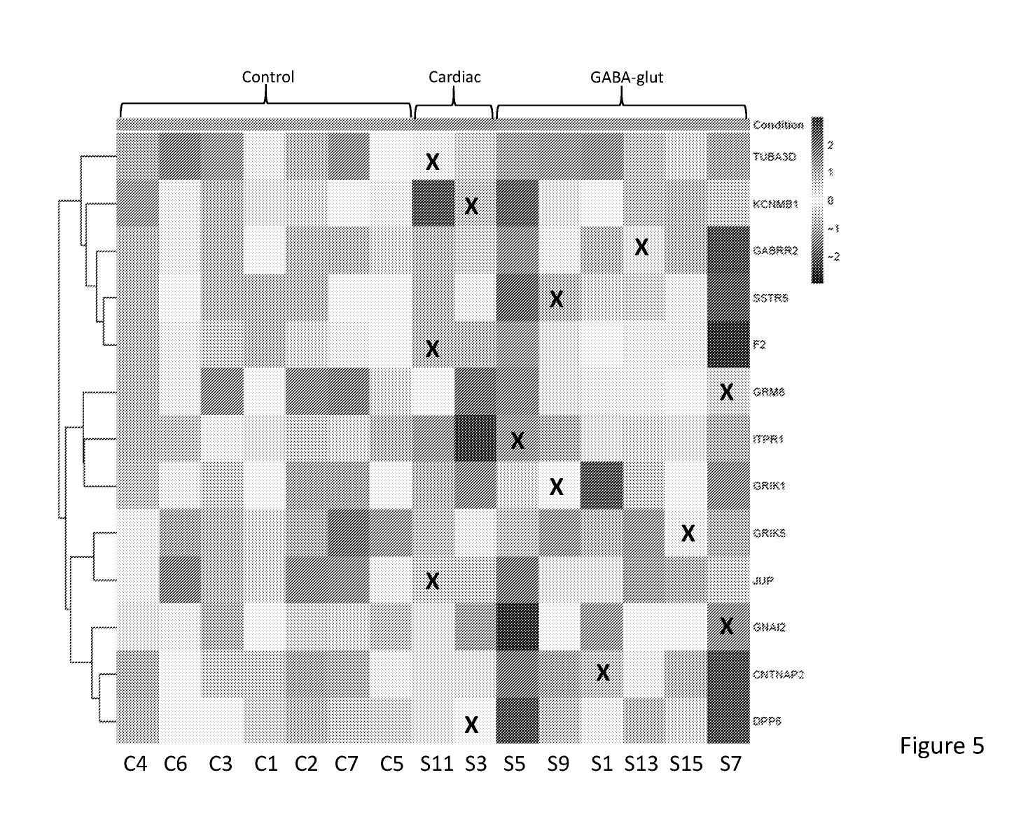

The mutations we have identified as predictive of a risk of developing SUDEP are specific. This is supported by our findings that SUDEP and Control patients (i.e., epilepsy patients who did not die of SUDEP) shared a large number (37) of somatic mutations, which were not unique to SUDEP phenotype (FIG. 4). Thus, while a combination of mutations might contribute to seizure development; specific predisposing mutations such as the ones we identified lead to increased risk of SUDEP.

The present disclosure provides a method for predicting an individual to be at risk of developing sudden unexpected death in epilepsy (SUDEP) comprising: a) obtaining a sample from the individual, said sample comprising cells (such as a blood sample); and b) sequencing nucleic acids from the sample to detect the presence or absence of one or more SUDEP specific mutations in one or more marker genes selected from the group consisting of: ITPR1, GABRR2, JUP, SSTR5, F2, KCNMB1, CNTNAP2, GRM8, GNAI2, TUBA3D, GRIK1, GRIK5 and DPP6. The SUDEP specific mutations are identified by their presence in the DNA from a population of individuals who had SUDEP, but absent in the DNA of from matched controls. The method of claim 1, wherein the mutation is detected at the DNA level. The specific mutation in the genes can be: a) for ITPR1, corresponding to nucleotide G at position 100 in SEQ ID NO:1 (such as change of G to A); b) for GABRR2, corresponding to nucleotide G at position 100 in SEQ ID NO: 2 (such as change of G to T); c) for JUP, corresponding to nucleotide A at position 100 in SEQ ID NO: 3 (such as change of A to G); d) for SSTR5, corresponding to nucleotide G at position 100 in SEQ ID NO: 4 (such as change of G to T); e) for F2, corresponding to nucleotide C at position 100 in SEQ ID NO: 5 (such as change of C to T); f) for KCNMB1, corresponding to nucleotide T at position 100 in SEQ ID NO: 6 (such as change of T to C); g) for CNTNAP2, corresponding to nucleotide G at position 100 in SEQ ID NO: 7 (such as change of G to A); h) for GRM8, corresponding to nucleotide A at position 100 in SEQ ID NO: 8 (such as change of A to G; i) for GNAI2, corresponding to nucleotide C at position 100 in SEQ ID NO: 9 (such as change of C to T); j) for TUBA3D, corresponding to nucleotide A at position 100 in SEQ ID NO: 10 (such as change of A to G); k) for GRIK1, corresponding to nucleotide A at position 100 in SEQ ID NO: 11 (such as change of A to G); l) for GRIK5, corresponding to nucleotide T at position 100 in SEQ ID NO: 12 (such as change of T to A); and m) for DPP6, corresponding to nucleotide C at position 100 in SEQ ID NO: 13 (such as change of C to G). The method may not comprise detecting mutations in the genes that were found to not be predictive, such as for example, the genes shown in FIG. 4.

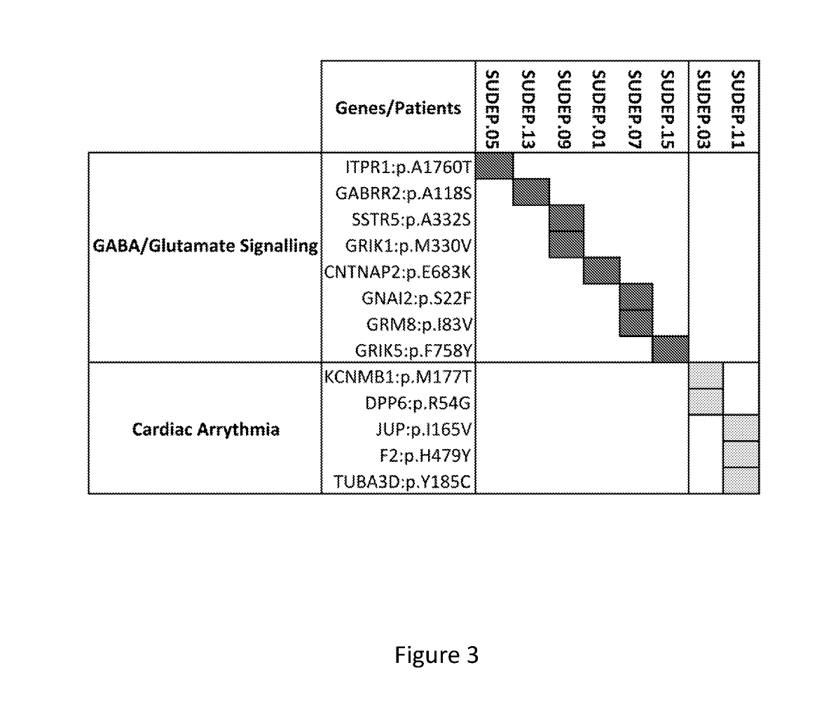

Our findings indicate that SUDEP patients can be divided into two groups, patients with mutations in Cardiac pathway genes (SUDEP Cardio) and GABA/Glutamate signaling (SUDEP Gaba/Glu) (FIG. 3). Since RNA sequencing data reveal that most ion channel genes are expressed in both brain and heart, albeit to markedly different degrees (e.g., SCN1A more in brain; SCN5A more in heart), mutations in a single gene can alter excitability in both myocardium (e.g., pacemaker, conduction, myocardium) and brain (e.g., cortex, brainstem).

To further support the notion that mutations identified herein (FIG. 3) are informative of an increased risk of SUDEP, we investigated if SUDEP patients had a distinct and distinguishable signature as compared to non-SUDEP patients. The results of this investigation are provided in Example 2. The expression of certain genes (Group 1) involved in Glutamate/GABA signaling shown in Table 3 was found to be generally increased over controls. The expression of certain other genes (Group 2) involved in Glutamate/GABA signaling was found to be generally decreased over controls. Expression of certain genes (Group 3) involved in cardiac function and regulation of blood pressure was found to be generally increased over controls, and expression of certain other genes (Group 4) was found to be decreased over controls. The genes of Group 1, Group 2, Group 3 and Group 4 are shown below in Tables 3, 4, 5 and 6 respectively. Our findings support that in addition to unique DNA mutations, SUDEP patients carry unique metabolic and functional signatures strengthening the link between previously identified DNA mutations and cell phenotype/function.

Based on the findings disclosed herein, this disclosure provides a method to obtain a signature predictive of the likelihood of progression to SUDEP. The signature can comprise two or more markers that are disclosed herein to be associated with SUDEP. The two or more markers may be from Group 1, Group 2, Group 3 or Group 4 or may be a combination of genes from these groups. The expression of any number of genes from each group, separately or simultaneously, may be determined. For example, the expression of from 1 to 47 genes in Group 1, from 1 to 45 in Group 2, from 1 to 40 in Group 3, and/or 1 to 41 in Group 4 can be determined.

TABLE 3
Group 1 Genes: Genes involved in Glutamate/GABA
signaling whose expression is increased in SUDEP

| No. | Gene | Chr:Start-End |
|---|---|---|
| 1 | LRRC71 | chr1:156920649-156933094 |
| 2 | SLC3A1 | chr2:44275459-44321494 |
| 3 | NUDT6 | chr4:122892643-122922606 |
| 4 | FAT4 | chr4:125316398-125492932 |
| 5 | SLC10A7 | chr4:146254931-146521933 |
| 6 | SOX30 | chr5:157625678-157652420 |
| 7 | GRM6 | chr5:178978326-178995123 |
| 8 | CFAP206 | chr6:87407982-87464465 |
| 9 | WISP3 | chr6:112054071-112070969 |
| 10 | COL10A1 | chr6:116118922-116126133 |
| 11 | PACRG | chr6:162727131-163315492 |
| 12 | LINC00574 | chr6:169790320-169802873 |
| 13 | PTCHD4 | chr6:47878027-48068689 |
| 14 | RP3-382I10.7 | chr6:87408011-87496140 |
| 15 | C7orf34 | chr7:142939505-142940868 |
| 16 | C8orf44-SGK3 | chr8:66667615-66860472 |
| 17 | ZMYND19 | chr9:137582078-137590490 |
| 18 | EBF3 | chr10:129835282-129963841 |
| 19 | UCMA | chr10:13221766-13234334 |
| 20 | LDHAL6A | chr11:18455883-18479600 |
| 21 | METTL12 | chr11:62665308-62668108 |
| 22 | C11orf21 | chr11:2297172-2301913 |
| 23 | OR52H1 | chr11:5544560-5545523 |
| 24 | GLIPR1L1 | chr12:75334682-75370560 |
| 25 | ZIC2 | chr13:99981771-99986773 |
| 26 | MIR4500 | chr13:87618664-87618740 |
| 27 | NOXRED1 | chr14:77394020-77423056 |
| 28 | CHRNA7 | chr15:32030497-32172521 |
| 29 | PLA2G4B | chr15:41837774-41848147 |
| 30 | B3GNT9 | chr16:67148104-67151214 |
| 31 | SLC16A6 | chr17:68267025-68291116 |
| 32 | ZNF519 | chr18:14103862-14132430 |
| 33 | TEAD2 | chr19:49340594-49362457 |
| 34 | ZNF749 | chr19:57435328-57445485 |
| 35 | TMEM221 | chr19:17435508-17448567 |
| 36 | SLC13A3 | chr20:46557832-46651440 |
| 37 | SOX18 | chr20:64047581-64049641 |
| 38 | SNHG11 | chr20:38446690-38450921 |
| 39 | RTEL1-TNFRSF6B | chr20:63659402-63698684 |
| 40 | DONSON | chr21:33577904-33588708 |
| 41 | CECR5-AS1 | chr22:17159398-17165445 |
| 42 | DRICH1 | chr22:23608451-23632321 |
| 43 | SLC5A4 | chr22:32218475-32255341 |
| 44 | A4GALT | chr22:42692120-42695633 |
| 45 | LINC00574 | chr6:65522-78075 |
| 46 | C7orf34 | chr7:962409-963911 |
| 47 | CHRNA7 | chr1:4315610-4454253 |

TABLE 4
Group 2 Genes: Genes involved in Glutamate/GABA
signaling whose expression is reduced in SUDEP

| No. | Gene | Chr:Start-End |
|---|---|---|
| 1 | ANXA5 | chr4:121667954-121697113 |
| 2 | APLNR | chr11:57233592-57237314 |
| 3 | BSCL2 | chr11:62690294-62706344 |
| 4 | C3 | chr19:6677703-6720682 |
| 5 | CD63 | chr12:55725481-55729707 |
| 6 | CHST7 | chrX:46573783-46598408 |
| 7 | CLPB | chr11:72292424-72434648 |
| 8 | COTL1 | chr16:84565593-84618077 |
| 9 | CSPG5 | chr3:47562238-47580792 |
| 10 | DOC2B | chr17:142788-181636 |
| 11 | F3 | chr1:94529224-94541800 |
| 12 | FABP7 | chr6:122779474-122784074 |
| 13 | GADD45A | chr1:67685060-67688338 |
| 14 | GLA | chrX:101397802-101407925 |
| 15 | GNB2L1 | chr5:181236936-181244209 |
| 16 | HLA-DRA | chr6:32439841-32445046 |
| 17 | HSPB11 | chr1:53921560-53945929 |
| 18 | IFT122 | chr3:129440253-129520339 |
| 19 | LGALS1 | chr22:37675607-37679806 |
| 20 | MIR4305 | chr13:39664033-39664135 |
| 21 | MIR4757 | chr2:19348428-19348505 |
| 22 | MIR553 | chr1:100281240-100281308 |
| 23 | MIR597 | chr8:9741671-9741768 |
| 24 | MMD2 | chr7:4905997-4959213 |
| 25 | MT2A | chr16:56608198-56609497 |
| 26 | NCAN | chr19:19211972-19252233 |
| 27 | PCDHGC5 | chr5:141489120-141512979 |
| 28 | PP7080 | chr5:470509-473098 |
| 29 | RNF181 | chr2:85595733-85597581 |
| 30 | S100A16 | chr1:153606885-153613083 |
| 31 | SAMM50 | chr22:43955420-43996533 |
| 32 | SCARA3 | chr8:27634180-27673020 |
| 33 | SDC3 | chr1:30873134-30908761 |
| 34 | SLC1A4 | chr2:64989400-65023865 |
| 35 | SNORA10 | chr16:1962333-1962466 |
| 36 | SNORA64 | chr2:30187433-30187566 |
| 37 | SNORA80B | chr2:10446713-10446849 |
| 38 | SNRPD2 | chr19:45687453-45692333 |
| 39 | SSR2 | chr1:156009047-156020959 |
| 40 | TP53RK | chr20:46684364-46689779 |
| 41 | TRAPPC1 | chr17:7930344-7931999 |
| 42 | TUBB2B | chr6:3224260-3227735 |
| 43 | UBC | chr12:124911603-124915348 |
| 44 | WLS | chr1:68125357-68232553 |
| 45 | ZDHHC18 | chr1:26826709-26857601 |

TABLE 5
Group 3 genes: Genes involved in Cardiac function
whose expression is increased in SUDEP

| No. | Gene | Chr:Start-End |
|---|---|---|
| 1 | ACAD11 | chr3:132558143-132660723 |
| 2 | APOM | chr6:31655470-31658210 |
| 3 | ASAP3 | chr1:23428562-23484178 |
| 4 | BPHL | chr6:3118691-3153578 |
| 5 | CCDC157 | chr22:30356634-30376829 |
| 6 | CHIC2 | chr4:54009788-54064690 |
| 7 | CISH | chr3:50606521-50611831 |
| 8 | CLDN3 | chr7:73768996-73770270 |
| 9 | CLEC2A | chr12:9913226-9932381 |
| 10 | CLIC3 | chr9:136994634-136996803 |
| 11 | COLCA2 | chr11:111298839-111308733 |
| 12 | CYP3A7 | chr7:99705043-99735096 |
| 13 | DRGX | chr10:49364180-49395451 |
| 14 | DYNLRB2 | chr16:80540956-80550644 |
| 15 | FAM182A | chr20:26054654-26086917 |
| 16 | GAPDHS | chr19:35533411-35545316 |
| 17 | IL23A | chr12:56338874-56340410 |
| 18 | LRRC61 | chr7:150323286-150338150 |
| 19 | METTL12 | chr11:62665308-62668108 |
| 20 | MIR101-2 | chr9:4850290-4850381 |
| 21 | MIR106B | chr7:100093992-100094074 |
| 22 | MIR1228 | chr12:57194503-57194576 |
| 23 | MIR1247 | chr14:101560286-101560422 |
| 24 | MIR30E | chr1:40754350-40754463 |
| 25 | MIR4669 | chr9:134379410-134379472 |
| 26 | MIR4672 | chr9:127869414-127869495 |
| 27 | MIR4689 | chr1:5862671-5862741 |
| 28 | MIR4753 | chr1:235190033-235190116 |
| 29 | MIR550A3 | chr7:29680733-29680828 |
| 30 | MIR99B | chr19:51692611-51692681 |
| 31 | NFKBID | chr19:35888240-35902303 |
| 32 | PDCD1 | chr2:241849880-241858908 |
| 33 | PGPEP1L | chr15:98968279-99005562 |
| 34 | PRTN3 | chr19:840959-848175 |
| 35 | RUNX3 | chr1:24899517-24929877 |
| 36 | SNORA5B | chr7:45105967-45106099 |
| 37 | SNORA70G | chr12:68627233-68627375 |
| 38 | STK32A | chr5:147234962-147387852 |
| 39 | TMEM129 | chr4:1715952-1721331 |
| 40 | ZBTB32 | chr19:35704526-35717038 |

TABLE 6
Group 4 genes: Genes involved in Cardiac function
whose expression is reduced in SUDEP

| No. | Gene | Chr:Start-End |
|---|---|---|
| 1 | ADTRP | chr6:11713523-11779170 |
| 2 | ARPIN | chr15:89895005-89912956 |
| 3 | BLM | chr15:90717326-90816165 |
| 4 | BRINP3 | chr1:190097661-190477882 |
| 5 | CCDC110 | chr4:185445181-185471759 |
| 6 | CD8B | chr2:86815556-86861915 |
| 7 | CDC20 | chr1:43358954-43363203 |
| 8 | CLDN6 | chr16:3014711-3020071 |
| 9 | CNBD2 | chr20:35968606-36030700 |
| 10 | DKFZP434I0714 | chr4:152536263-152539263 |
| 11 | GDPD4 | chr11:77216557-77287418 |
| 12 | GEMIN6 | chr2:38778184-38785000 |
| 13 | GPR18 | chr13:99254731-99261744 |
| 14 | GRIK3 | chr1:36795526-37034125 |
| 15 | HGH1 | chr8:144137768-144140843 |
| 16 | HSPB3 | chr5:54455600-54456384 |
| 17 | HTR1D | chr1:23191894-23194729 |
| 18 | IRGM | chr5:150846522-150848669 |
| 19 | MPEG1 | chr11:59208509-59212951 |
| 20 | NUSAP1 | chr15:41332870-41380402 |
| 21 | PCP4L1 | chr1:161258726-161285450 |
| 22 | PIF1 | chr15:64815631-64825668 |
| 23 | PP12613 | chr4:121764584-121766814 |
| 24 | PRKG2 | chr4:81087369-81215117 |
| 25 | PSTK | chr10:122980039-122990390 |
| 26 | RGPD3 | chr2:106404989-106468376 |
| 27 | SNAI2 | chr8:48917767-48921740 |
| 28 | SNORD115-39 | chr15:25241745-25241827 |
| 29 | SNORD126 | chr14:20326449-20326526 |
| 30 | SNORD2 | chr3:186784795-186784864 |
| 31 | SNORD2 | chr10:56595962-56596031 |
| 32 | SNORD31 | chr13:107320894-107320963 |
| 33 | SPINK8 | chr3:48306841-48328341 |
| 34 | SRD5A1 | chr5:6633342-6674386 |
| 35 | STARD5 | chr15:81309052-81324125 |
| 36 | TMPRSS11D | chr4:67820875-67884032 |
| 37 | TPK1 | chr7:144451940-144836053 |
| 38 | TRIM34 | chr11:5619763-5644398 |
| 39 | TSPAN1 | chr1:46175086-46185958 |
| 40 | WNT2 | chr7:117276630-117323289 |
| 41 | ZNF845 | chr19:53333748-53354869 |

These markers can be used to assess risk of an individual to develop SUDEP. The risk assessment can be done on a continuum. For example, if all markers from Group 1 or Group 3 are found to be highly expressed (such as, for example, assigned a score of +2), the individual may be considered at higher risk than an individual who shows enhanced expression of fewer than all the markers, or in whom the expression is not so highly enhanced. Similarly, if the expression of all the markers from Group 2 or Group 4 is reduced (such as, for example, assigned a score of −2), then the individual is considered at higher risk than an individual who shows reduced expression of fewer than all the genes or shows less decrease in expression.

The sample in which the determination is carried out can be any tissue in which these genes are expressed or any fluid where brain cell RNA is excreted into. For example, a convenient tissue is brain tissue or brain cells obtained floating from the cerebrospinal fluid during a routine spinal tap procedure. A biopsy of the brain tissue can be obtained during any surgical procedure carried out or can be obtained during a procedure intended to collect a biopsy specimen. Furthermore, RNA from brain tissue can also be extracted from exosomes circulating in the blood or in urine using established protocols (See e.g., Li et al., Philos Trans R Soc Lond B Biol Sci. 2014 Sep. 26; 369(1652): 20130502. doi: 10.1098/rstb.2013.0502 PMCID: PMC4142023). The expression level of at least one marker is determined in the sample. For example, the expression level of at least one marker from Group 1, Group 2, Group 3, or Group 4 can be determined.

Based on the findings provided in Example 2, the expression levels of one or more genes set forth in Group 1, 2, 3 and/or 4 can be determined and compared to a reference (also referred to herein as control). The reference levels may be the levels from epilepsy patients who were not afflicted with SUDEP. The expression of the genes can be used to generate a reference pattern, which can then be used to estimate the likelihood of progression to SUDEP.

The expression of more than one marker from a Group or from each Group can be determined. For example, the expression of at least two markers from a group or at least two groups can be determined. For example, a finding of an enhanced expression of at least one gene from Group 1, reduced expression from at least one gene in Group 2, enhanced expression of at least one gene in Group 3, and/or reduced expression of at least one gene in Group 4 can be predictive of likelihood of progression to SUDEP. The expression of more than one gene up to all the genes from each or all groups can be determined.

The markers provided in this disclosure show a sufficient difference in expression from SUDEP groups to controls to use them as classifiers for the likelihood of progression. Thus, comparison of an expression pattern from a signature to another expression pattern from another signature may indicate and inform a change in the expression of genes in the brain, and likelihood of progression to SUDEP. Additionally, or alternatively, changes in intensity of expression may be scored, either as increases or decreases. Any significant change can be used. Typical changes which are more than 1-fold or 2-fold are suitable for use.

Some methods provided in this disclosure relate to diagnostic or prognostic uses of information about expression levels. For example, expression patterns from signatures can be obtained. For example, the disclosure provides a method of determining an expression pattern, comprising collecting a suitable biological sample comprising cells (such as brain cells), determining the expression level of more than one marker in the sample, said marker being selected from gene in Tables 3 to 6, and obtaining an expression pattern for the signature. The expression pattern, as a whole, or for individual genes or groups of genes, can be compared to similar expression patterns generated from controls.

The groups of genes that can be used in the present methods are those whose expression is specifically associated with SUDEP. For example, genes involved with Glutamate/GABA signaling, and or genes involved with regulation of blood pressure and heart development can be used. Based on the disclosure provided herein, other genes may be identified whose expression is predictive of progression to SUDEP.

Expression of genes can be detected by techniques well known in the art. For example, mRNA can be detected from the cells and/or expression products such as peptides and proteins can be detected, or whole transcriptome analysis (RNA sequencing) can be carried out. Detection of mRNA can involve sample extraction, PCR amplification, nucleic acid fragmentation and labeling, extension reactions, and transcription reactions. Methods of isolating total RNA are well known to those of skill in the art. For example, total nucleic acid is isolated from a given sample using, for example, an acid guanidinium-phenol-chloroform extraction method and polyA selection for mRNA using oligo dT column chromatography or by using beads or magnetic beads with (dT)n groups attached (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987)).

Microarray technology can be used to evaluate expression status of a plurality of genes. Sequence based techniques, like serial analysis of gene expression (SAGE, SuperSAGE) are also used for gene expression profiling. In an mRNA or gene expression profiling microarray, the expression levels of multiple genes can be simultaneously evaluated. For example, microarray-based gene expression profiling can be used to obtain gene signatures of individuals suspected of being as risk of SUDEP.

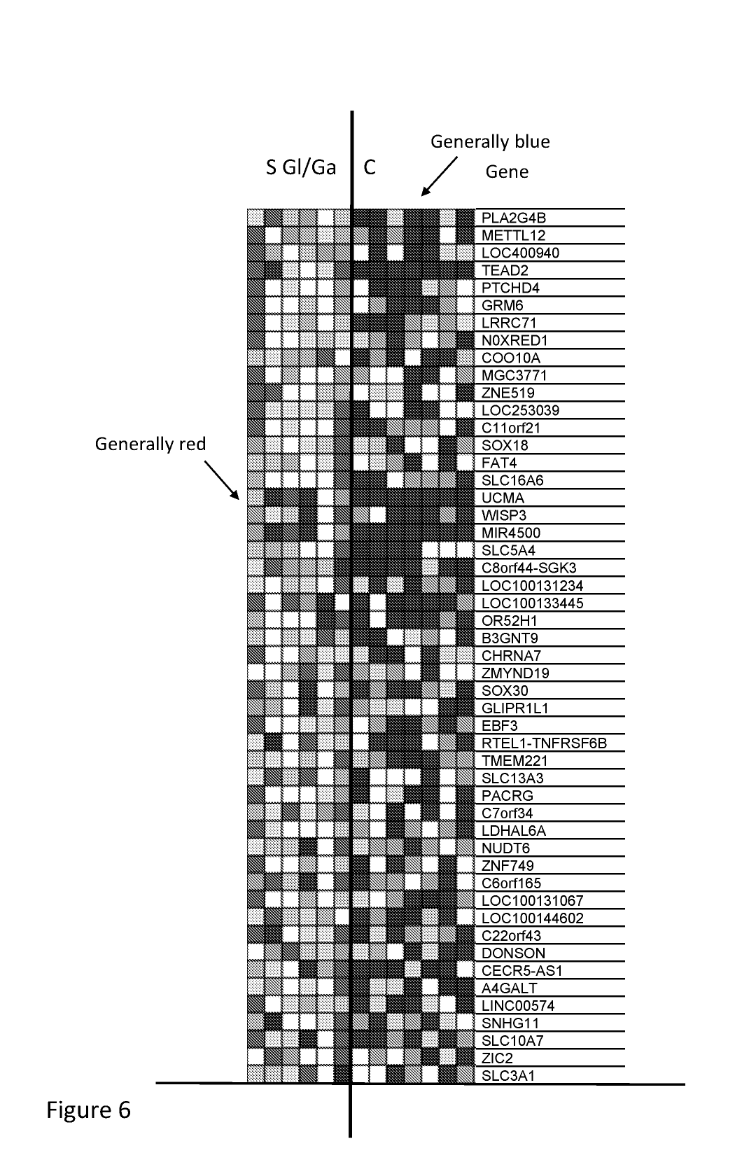

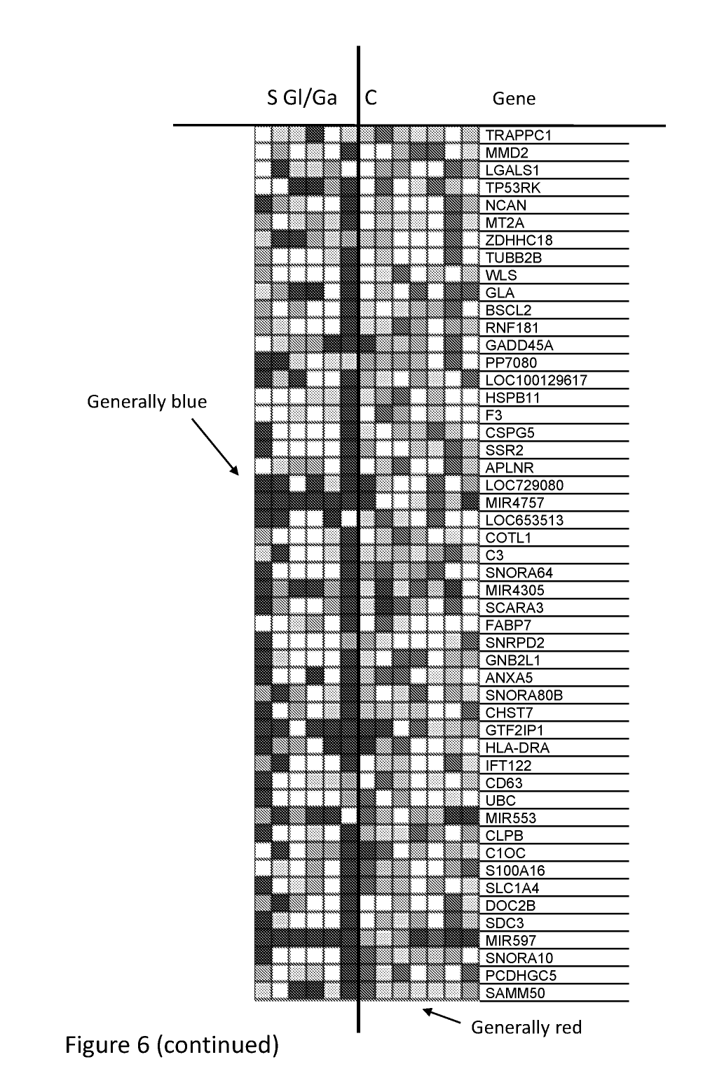

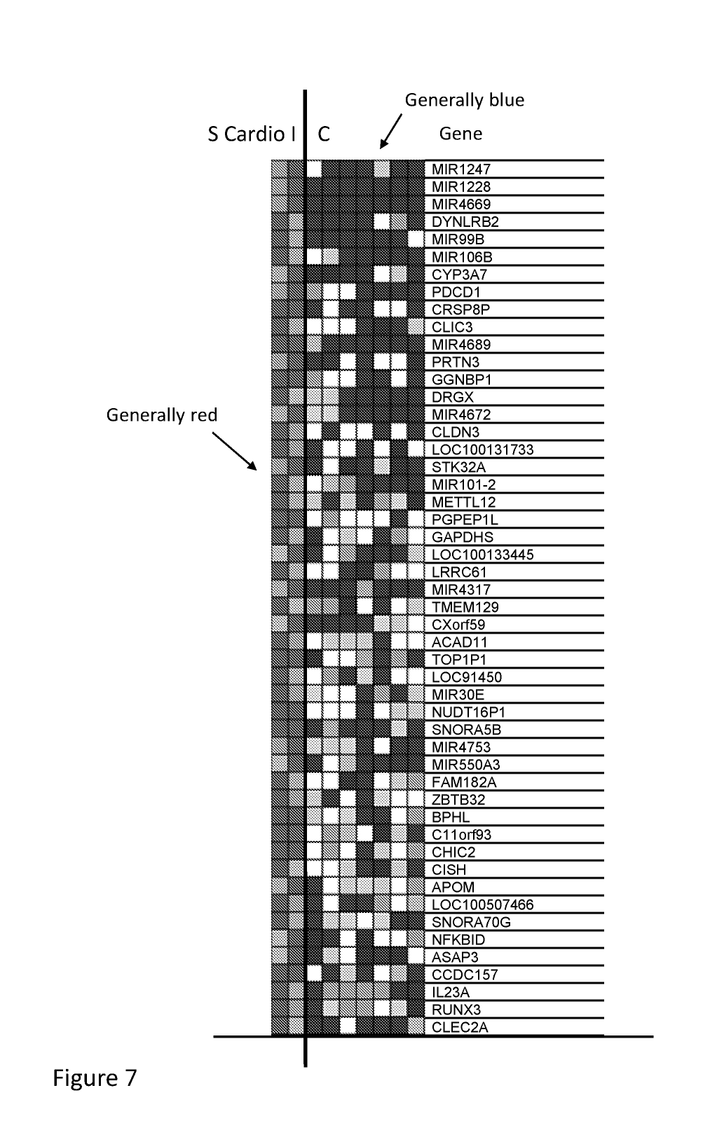

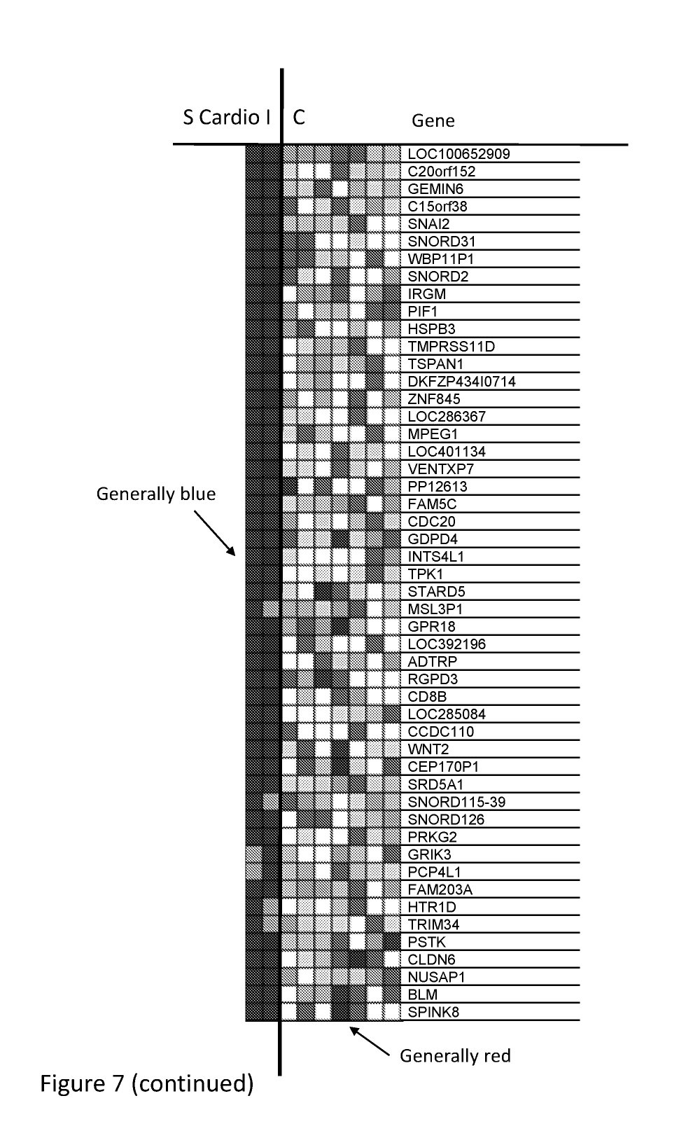

This disclosure also provides a SUDEP tool or kit, which can be used for determining the likelihood of individuals to progress to SUDEP. The tool can comprise one or more of reagents for performance of transcriptome analysis, charts providing patterns of expression of markers as identified here and instructions and/or guidance for interpretation of results. For example, the charts may be similar to FIGS. 6 and/or 7, which provide an indication of which genes may exhibit enhanced expression and which genes may exhibit reduced expression.

The invention is further described in the examples provided below, which intended to illustrate the invention and not intended to be restrictive.

### Example 1

Methods

Whole Exome Sequencing

DNA was isolated from 8 SUDEP and non-SUDEP (i.e. Control) patients' formalin fixed paraffin embedded brain tissue which was previously resected during the brain surgery for epilepsy management. 250 ng of DNA from each sample was sheared to an average of 150 bp in a Covaris instrument for 360 seconds (Duty cycle—10%; intensity—5; cycles/Burst—200). Barcoded libraries were prepared using the Kapa Low-Throughput Library Preparation Kit (Kapa Biosystems), amplified using the KAPA HiFi Library Amplification kit (Kapa Biosystems) (8 cycles) and quantified using Qubit Fluorimetric Quantitation (Invitrogen) and Agilent Bioanalyzer. An equimolar pool of the 4-barcoded libraries (300 ng each) was used as an input to capture the exome using one reaction tube of the Nimblegen SeqCap EZ Human Exome Library v3.0 (Roche, cat #06465684001), according to the manufacturer's protocol. The pooled capture library was quantified by Qubit (Invitrogen) and Bioanalyzer (Agilent) and sequenced on an Illumina HiSeq 2500 using a paired end, 100 nucleotides in length run mode, to achieve an average of 100× coverage.

Exome Bioinformatics (Variant Analysis)

Demultiplexed fastq reads were aligned to the hg19 genome build (GRCh37) using the Burrows-Wheeler Aligner (BWA) (Li et al., Bioinformatics 25, 1754-1760 (2009). Further indel realignment, base-quality score recalibration and duplicate-read removal were performed using the Genome Analysis Toolkit (GATK) v2.4-9². GATK Haplotype Caller (McKenna, A. et al. Genome Res. 20, 1297-1303 (2010) was used to generate single-nucleotide variation (SNV) and indel calls using standard, default parameters. SNV's found in the living epilepsy controls as well as germline variants found in the 1000 Genomes Project (1000 Genomes Project Consortium. Nature 467, 1061-1073 (2010), ESP5400 (National Heart, Lung, and Blood Institute (NHLBI) GO Exome Sequencing Project) and dbSNP132 (Sherry et al., Nucleic Acids Res. 29, 308-311 (2001) were excluded. Resulting putative mutations were annotated based on RefSeq (Release 55) using Annovar (Wang et al., Nucleic Acids Res. 38, e164 (2010)) and only the missense and nonsense mutations were retained. The mutated genes were queried for pathways using Ingenuity Pathway Analysis (IPA) tool, which identified 5 genes that were associated with Cardiac Arrhythmia (see below). Further, the mutations were examined for functional consequence using Ingenuity Variant Analysis (IVA) software. This analysis revealed 8 genes in GABA/Glutamate receptor signaling pathways (details below). All 13-candidate mutations were manually inspected via Integrative Genomics Viewer (IGV) v2.1 (Robinson, J. T. et al. Integrative genomics viewer. Nat. Biotechnol. 29, 24-26 (2011)).

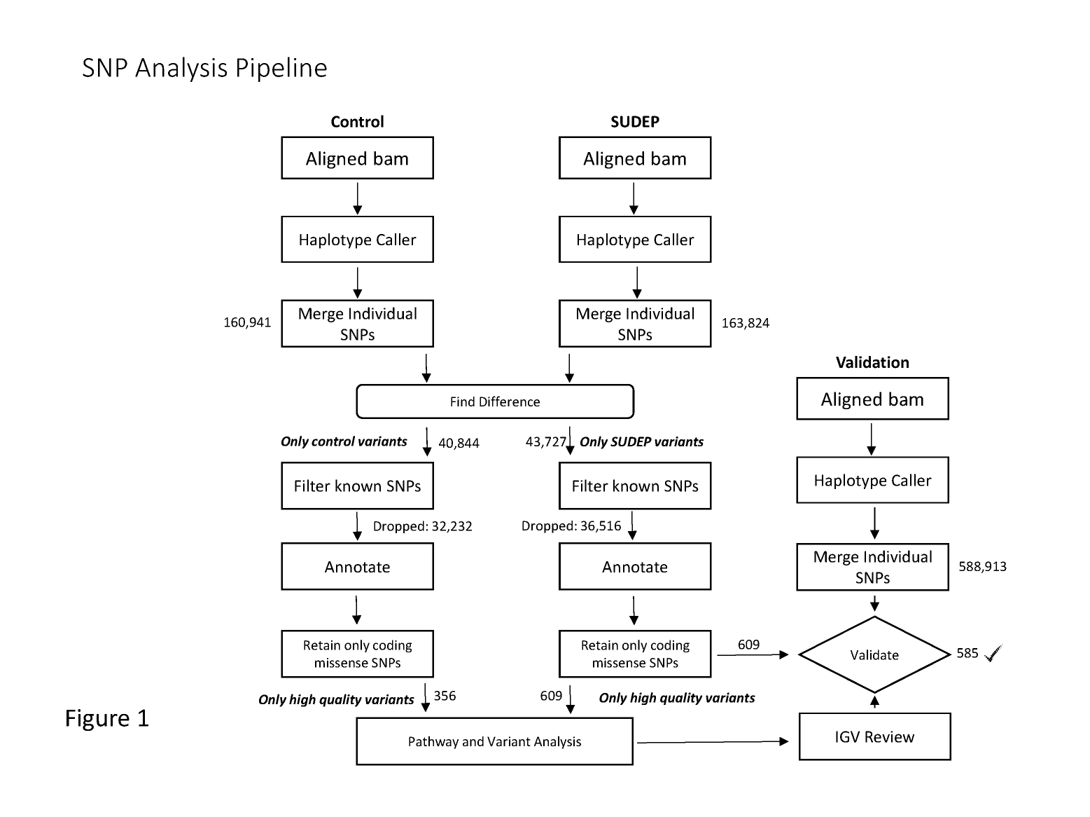

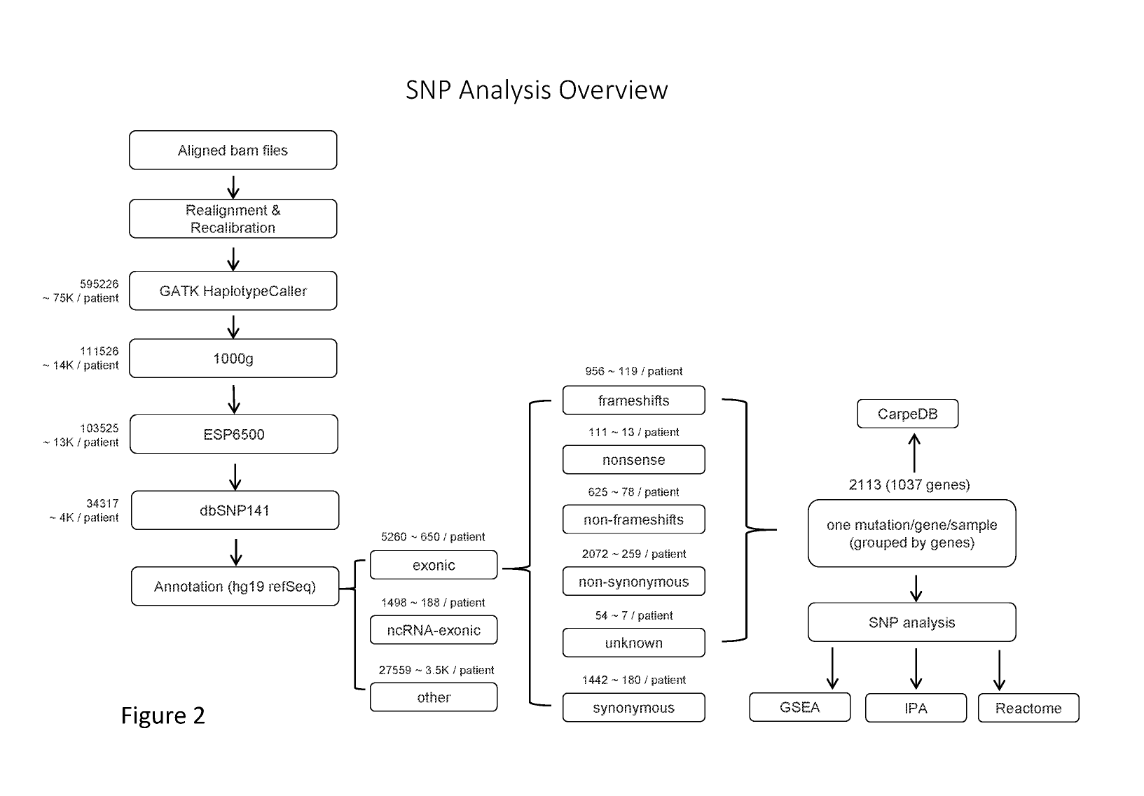

The analysis is shown in FIGS. 1 and 2. The following specific mutations were identified uniquely in the SUDEP patient population (FIG. 3):

Genes/Mutations Associated with Cardiac Arrhythmia:
KCNMB1: calcium-activated potassium channel subunit beta-1. The mutation is at chromosome 5, position: 169805754; and the amino change is p.M177T.
DPP6: dipeptidyl aminopeptidase-like protein 6. The mutation is at chromosome 7, position: 153750065; and the amino acid change is p.R54G.
JUP: junction plakoglobin. The mutation is at chromosome 17, position: 39925435; and the amino acid change is p.I165V.
F2: thrombin. The mutation is at chromosome 1, position: 46750350 and the amino acid change is p.H479Y.
TUBA3D: tubulin 3D. The mutation is at chromosome 2, position: 132237820 and the amino acid change is p.Y185C.

Genes/Mutations Associated with GABA/Glutamate Pathway:
ITPR1: inositol 1,4,5-triphosphate receptor. The mutation is at chromosome 3, position 4776961 and the amino acid change is p.A1760T.
GABRR2: Gamma-aminobutyric acid receptor Rho2 subunit. The mutation is at chromosome 6, position 89978890 and the amino acid change is p.A118S.
SSTR5: somatostatin receptor 5. The mutation is at chromosome 16, position 1129862 and the amino acid change is p.A332S.
CNTNAP2: contactin-associated protein-like 2. The mutation is at chromosome 7, position 147336347 and the amino acid change is p.E683K.
GRM8: metabotropic glutamate receptor 8. The mutation is at chromosome 7, position 126883012 and the amino acid change is p.I83V.
GNAI2: guanine nucleotide-binding protein G(I), alpha-2 subunit. The mutation is at chromosome 3, position 50264620 and the amino acid change is p.S22F.
GRIK1: glutamate receptor, inotropic kainate 1. The mutation is at chromosome 21, position 31015256 and the amino acid change is p.M330V.
GRIK5: glutamate receptor, inotropic kainate 5. The mutation is at chromosome 19, position 42507826 and the amino acid change is p.F758Y.

### Example 2

Since there are currently no models that would allow us to reliably test functional cumulative effect of mutations we identified in our cohorts, we set to perform a whole transcriptome analysis to obtain an independent confirmation that brains of SUDEP patients are distinctly different than non-SUDEP epilepsy patients (Controls). We performed whole transcriptome analysis (RNA sequencing) of the same brain tissue samples on which we performed whole exome DNA sequencing to identify mutations.

We carried out experiments to determine whether mutations in SUDEP specific genes are associated with distinct changes in expression of the mutant gene and/or signaling family (Cardiac vs GABA/Glutamate signaling), whether SUDEP patients have unique gene expression signature that distinguishes them from Control patients; and if specific enrichment of gene groups that would be associated with SUDEP phenotype by performing Gene Set Enrichment Analysis (GSEA).

As shown in FIG. 3, we observed that targeted analysis of SUDEP mutated genes in comparison with Controls did not show clear up/down regulation of mutated genes albeit overall SUDEP patients seemed to have more extreme changes of expression than Controls. This is not surprising as we postulated that mutations have an effect on the function of the genes rather than level of expression and we did not expect that mutation would lead to complete loss of the protein.

We first separated SUDEP GABA/Glutamate (S Gl/Ga, FIG. 4) and SUDEP Cardiac (S Cardio, FIG. 6) patients and compared them to Controls individually. In both analyses, SUDEP patients had distinct gene expression signature when compared to Controls (Comparing TOP 50 most differentially expressed genes for each group). Despite the fact that both groups (SUDEP and Controls) of patients carry the same initial clinical diagnosis (epilepsy) on gene expression level they appear as two distinct diseases. Of note, the design of our study was such to minimize the effect of potential bias due to other factors. Therefore, both SUDEP and non-SUDEP groups were matched for age at surgery (median 37 and 34 years, respectively) and age of seizure onset (median 13 and 10 years respectively). Patients were also matched for post operative clinical outcome, one of the SUDEP patients and only two of non-SUDEP seizure controls were free of seizures after the surgery. Median survival from surgery to death was 5.5 years in SUDEP patients (range, 1-11 years) and median follow up of non-SUDEP patients was 11 years (range, 1-12 years). Therefore we concluded that SUDEP patients have distinct gene expression profile and assume that it is due to the underlying unique gene mutations.

To identify functional effect of the SUDEP genotype, we performed GSEA. In SUDEP GABA/Glut we identified enrichment of genes associated with sugar metabolism, sugar binding and oxygen binding. Sugar is a critical brain metabolite and abnormal sugar metabolism, inability to bind could be detrimental during seizures when the need of sugar increases in brain cells. Similarly, oxygen is critical for brain metabolism and abnormalities in oxygen metabolism can be fatal during the seizures. We further identified enrichment of genes associated with regulation of blood pressure and heart development further strengthening the association between the epilepsy and heart function for risk of SUDEP. Lastly, we identified enrichment of genes associated with drug metabolism. One of the well-known risks of SUDEP is inability to control seizures by medication. Patients with hypermetabolism of antiepileptic drugs would likely have shorter lifespan of medication in their system and therefore higher risk of developing sudden, potentially fatal seizure event. The SUDEP Cardiac cohort was smaller (2 patients). However even in this cohort, we were able to identify the enrichment for genes associated with higher risk of diabetes, particularly type 1.

Methods:

Nucleic Acids (DNA, RNA) Extraction

DNA and RNA were extracted from the formalin fixed paraffin embedded surgical pathology brain tissue using automated Maxwell Promega system per manufacturer's protocols.

DNA Sequencing

Whole exome DNA sequencing was performed using SeqCap capture (NimbleGen) and 50 base-pair paired-end sequencing. Exome sequencing. 250 ng of DNA from each sample were sheared on a Covaris instrument for 360 seconds (Duty cycle—10%; intensity—5; cycles/Burst—200). Barcoded libraries were prepared using the Kapa Low-Throughput Library Preparation Kit Standard (Kapa Biosystems), amplified using the KAPA HiFi Library Amplification kit (Kapa Biosystems) (8 cycles) and quantified using Qubit Fluorimetric Quantitation (Invitrogen) and Agilent Bioanalyzer. An equimolar pool of the 4 barcoded libraries (300 ng each) were used as input to capture the exome using one reaction tube of the Nimblegen SeqCap EZ Human Exome Library v3.0 (Roche, cat #06465684001), according to the manufacturer's protocol. The pooled capture library was quantified by Qubit (Invitrogen) and Bioanalyzer (Agilent) and sequenced on an Illumina Illumina HiSeq 2500 using a paired end, 100 nucleotides in length run mode, to achieve an average of 100× coverage.

DNA Sequencing Analysis

Realigned exomes were queried for SNPs using HaplotypeCaller (GATK). High frequency SNPs found in 1000 g, ESP6500 and dbSNP141 were filtered out. Resulting filtered putative mutations were annotated using ANNOVAR RefSeq hg19. Synonymous mutations were excluded. Mutations were grouped by genes and analyzed using MSigDB, IPA, Reactome and CarpeDB databases. Ingenuity™ pathway (IPA) and variant analysis (IVA; ingenuity.com) was performed to identify candidate mutations involved in cardiac and central nervous system function.

RNA Sequencing

Whole transcriptome analysis was performed. RNASeq libraries were prepared using the Clontech SMARTer Stranded Total RNA-Seq Kit library prep, with Ribozero Gold to remove rRNA, the recommended input ranging from 250 pg to 10 ng of total mammalian RNA, following the manufacturer's protocol. The libraries were pooled equimolarly, and loaded on high output Illumina HiSeq 2500 flow cells, using v4 reagents, as paired 50 nucleotide reads. Libraries were pooled and distributed uniformly across 3 lanes in order to generate 60-80 million reads per sample. Following this approach, we are able to prepare high quality libraries and perform sequencing. The alignment statistics were optimal with high concordant pair alignment rates and low multiple alignment rates.

RNA-Seq Data Analysis

Raw sequencing data were received in FASTQ format. Read mapping was performed using Tophat 2.0.9 against the hg19 human reference genome. The resulting BAM alignment files were processed using the HTSeq 0.6.1 python framework and respective hg19 GTF gene annotation, obtained from the UCSC database. Subsequently, the Bioconductor package DESeq2(3.2) was used to identify differentially expressed genes (DEG). This package provides statistics for determination of DEG using a model based on the negative binomial distribution. The resulting values were then adjusted using the Benjamini and Hochberg's method for controlling the false discovery rate (FDR). Genes with an adjusted p-value <0.05 were determined to be differentially expressed. Gene Set enrichment analysis was performed utilizing GSEA v.2.2.2.

1. A method for predicting an individual to be at risk of developing sudden unexpected death in epilepsy (SUDEP) comprising:
a) obtaining a sample from the individual, said sample comprising cells; and
b) sequencing nucleic acids from the sample to detect the presence or absence of one or more SUDEP specific mutations in one or more marker genes selected from the group consisting of: ITPR1, GABRR2, JUP, SSTR5, F2, KCNMB1, CNTNAP2, GRM8, GNAI2, TUBA3D, GRIK1, GRIK5 and DPP6, wherein the SUDEP specific mutations are identified by their presence in the DNA from a population of individuals who had SUDEP, but absent in the DNA of from matched controls.

2. The method of claim 1, wherein the mutation is detected at the DNA level.

3. The method of claim 1, wherein the specific mutation in the genes comprises:
a) for ITPR1, corresponding to nucleotide G at position 100 in SEQ ID NO:1;
b) for GABRR2, corresponding to nucleotide G at position 100 in SEQ ID NO: 2;
c) for JUP, corresponding to nucleotide A at position 100 in SEQ ID NO: 3;
d) for SSTR5, corresponding to nucleotide G at position 100 in SEQ ID NO: 4;
e) for F2, corresponding to nucleotide C at position 100 in SEQ ID NO: 5;
f) for KCNMB1, corresponding to nucleotide T at position 100 in SEQ ID NO: 6;
g) for CNTNAP2, corresponding to nucleotide G at position 100 in SEQ ID NO: 7;
h) for GRM8, corresponding to nucleotide A at position 100 in SEQ ID NO: 8;
i) for GNAI2, corresponding to nucleotide C at position 100 in SEQ ID NO: 9;
j) for TUBA3D, corresponding to nucleotide A at position 100 in SEQ ID NO: 10;
k) for GRIK1, corresponding to nucleotide A at position 100 in SEQ ID NO: 11;
l) for GRIK5, corresponding to nucleotide T at position 100 in SEQ ID NO: 12;
m) for DPP6, corresponding to nucleotide C at position 100 in SEQ ID NO: 13.

4. (canceled)

5. (canceled)

6. The method of claim 1, wherein if the individual is identified as having one or more SUDEP specific mutations in the genes ITPR1, GABRR2, SSTR5, CNTNAP2, GRM8, GNAI2, GRIK1 or GRIK5, then the individual is further administered a gamma aminobutyric acid (GABA) receptor agonist, GABA reuptake inhibitor, a GABA transaminase inhibitor, or a glutamate blocker.

7. A panel comprising two or more probes that can detect two or more mutations recited in claim 3.

8. The panel of claim 7, wherein the probes are affixed to a substrate and are detectably labeled.

9. (canceled)

10. (canceled)

11. (canceled)

12. (canceled)

13. (canceled)


SEQUENCE LISTING (13 sequences)

SEQ ID NO: 1 - DNA, 200 bp, human
tccagctctatgagcaggggtgagatgagtctggccgaggttcagtgtcaccttgacaaggagggggcttccaatctagttatcgacctcatcatgaacgcatccagtgaccgagtgttccatgaaagcattctcctggccattgcccttctggaaggaggcaacaccaccatccaggtaggaaggcagcttggctactg

SEQ ID NO: 2 - DNA, 200 bp, human
tgtcatgagtgaacgatcttttggagtgaacaaagaagacatcagggacccagatcttcttcaccagccggccatcgaaggtcatgctcttgttgctggcgctggagaaagctagcctctcatccttccagtaatgccgcaggtacagggtcatagtgaagtcctgtgggagccggggtgagaccagacaaaaatggctt

SEQ ID NO: 3 - DNA, 200 bp, human
cgctggtattctgcatggtacgcacgacagcggccaccagctggggcgagcccatcagggcccgccgcgacgcctccttcttcgacagctggttcacaatcatggccgccttggtcaccaccacctggagggcaaaggcaggggcggggacgtgagcactaaggagaggccgggatacccttccacagagctgaggaggg

SEQ ID NO: 4 - DNA, 200 bp, human
gccaaccccgtcctctacggcttcctctctgacaacttccgccagagcttccagaaggttctgtgcctccgcaagggctctggtgccaaggacgctgacgccacggagccgcgtccagacaggatccggcagcagcaggaggccacgccacccgcgcaccgcgccgcagccaacgggcttatgcagaccagcaagctgtg

SEQ ID NO: 5 - DNA, 200 bp, human
aagatctacatccaccccaggtacaactggcgggagaacctggaccgggacattgccctgatgaagctgaagaagcctgttgccttcagtgactacattcaccctgtgtgtctgcccgacagggagacggcagccaggtgggccaccagatgcttgttagctgaggggcagaagccaagttctgggcctggctctgatac

SEQ ID NO: 6 - DNA, 200 bp, human
ggcccagccagtcccctgtgccctgacaagtggtatggcatggatggatggctctacttctgggccgccaggatggacaggtactggttgctcttcaccatggcgataatgaggaggccaccggtcagcaggaaggtgggccagaagagggagaagaggagggcctggggcccgtagaggcgctggaataggacgctggt

SEQ ID NO: 7 - DNA, 200 bp, human
gtggtcggctacaacccagaaaaatactcagtgacacagctcgtttacagcgcctccatggaccagataagtgccatcactgacagtgccgagtactgcgagcagtatgtctcctatttctgcaagatgtcaagattgttgaacaccccaggtaggctgagaatggaatgttacttttaatcactatctcagctggtgct

SEQ ID NO: 8 - DNA, 200 bp, human
actgctccaaagcataggtgtccctagagcacgtgtcgaggatgcggacacccagagtgatgttggaaaggagatcagggtccttgttaatctggtcaattgcataaagcatggcctccagtctgtgaatccccttttccttcttcagctccccacaaggcacccctctctctccctttgcgtggacagggaagagaccc

SEQ ID NO: 9 - DNA, 200 bp, human
tgtgaagtggaagcgcgagaaggagggagcgtctcatgacggagggtgtgaagacgctaggctggacgaagcagaaaggcgggtgtcactggggacgttctgagggtaagccgatggcggctatcgcggaggagaccctggcgaggtggggccccgcgcggggcaagggggatggggtgccacagagggctagttgcaag

SEQ ID NO: 10 - DNA, 200 bp, human
tgctcatggagcggctctcagtggattacggcaagaagtccaagctagagtttgccatttacccagccccccaggtctccacagccgtggtggagccctacaactccatcctgaccacccacacgaccctggaacattctgactgtgccttcatggtcgacaatgaagccatctatgacatatgtcggcgcaacctggac

SEQ ID NO: 11 - DNA, 200 bp, human
ggttcataaatctgggtccgaggcgccatggcttatgtctatggcactgcagggagctgacggtcagctgggatgcccggtgcgaggcaatggccaccatgtacacagcatcgtacatcagagccgcttcagtctgtggaggaaaacacacaccgcatcttaaattccacttttgcttaccttcctttacttgcataatc

SEQ ID NO: 12 - DNA, 200 bp, human
ggaaggccgtggtgagggcacagtttggggtttggggcggtcagggctgcagggcccgatggctggtccagcccctcgtgtgcctgcccaggctccccgttccgggatgagatcacactggccatcctgcagcttcaggagaacaaccggctggagatcctgaagcgcaagtggtgggaggggggccggtgccccaagga

SEQ ID NO: 13 - DNA, 200 bp, human
cccccggaggcgagtcacctcctgggcggccaggggcccgaggaggacggcggcgcaggagccaagcccctcggcccgcgggcgcaggcggcggcgccccgggagcgcggcggcggcggcggcggcgcgggtggccggccccggttccagtaccaggcgcggagcgatggtgacgaggaggacgtaagagcttctcgggg